(19) United States
(12) Patent Application Publication
(10) Pub. No.: US 2010/0209944 A1
(43) Pub. Date: Aug. 19, 2010

(54) ADAMTS-7 AS A BIOMARKER FOR CANCERS OF EPITHELIAL ORIGIN

(76) Inventors: Marsha A. Moses, Brookline, MA (US); Roopali Roy, Newton, MA (US)

Correspondence Address: WOLF GREENFIELD & SACKS, P.C., 600 ATLANTIC AVENUE, BOSTON, MA, 02210-2206, US

(21) Appl. No.: 12/555,763

(22) Filed: Sep. 8, 2009

Related U.S. Application Data
(63) Continuation of application No. 11/815,821, filed on Mar. 13, 2008, now abandoned.
(60) Provisional application No. 60/653,818, filed on Feb. 17, 2005.

(51) Int. Cl. G01N 33/53; G01N 33/566; B01L 3/00

(52) U.S. Cl. 435/7.9; 435/7.1; 436/501; 422/61

(57) ABSTRACT

ADAMTS-7 expression and activity are up regulated in patients that have cancers of epithelial origin. Accordingly, the present invention is directed to methods diagnosis of cancers of epithelial origin (e.g. breast cancer, prostate cancer, bladder cancer, brain cancer and hepatic cancer). In particular, the presence of ADAMTS-7 in a biological sample is indicative of cancer of epithelial origin. Thus, measuring the level of ADAMTS-7 in biological samples (e.g. urine or blood) provides a quick, easy, and safe screen that can be used to diagnose cancer in a patient.

### CROSS-REFERENCE TO RELATED APPLICATIONS

This application is a continuation of patent application Ser. No. 11/815,821 filed Feb. 13, 2006, which is a national stage of PCT/US2006/004985 filed Feb. 13, 2006, which claims the benefit under 35 U.S.C. §119(e) of U.S. provisional Patent Application No. 60/653,818 filed Feb. 17, 2005, the disclosures of which are incorporated herein by reference.

### FIELD OF THE INVENTION

The present invention relates to methods for the diagnosis and prognosis of cancers of epithelial origin by assessing levels of ADAMTS-7 in a biological sample obtained from a patient.

### BACKGROUND OF THE INVENTION

One of the most important factors in the survival of cancer is detection at an early stage. Clinical assays that detect the early events of cancer offer an opportunity to intervene and prevent cancer progression. With the development of gene profiling and proteomics there has been significant progress in the identification of molecular markers or “biomarkers” that can be used to diagnose and prognose specific cancers. For example, in the case of prostate cancer, the antigen PSA (for prostate specific antigen) can be detected in the blood and is indicative of the presence of prostate cancer. Thus, the blood of men at risk for prostate cancer can be quickly, easily, and safely screened for elevated PSA levels.

Even though there has been significant progress in the field of cancer detection, there still remains a need in the art for the identification of new biomarkers for a variety of cancers that can be easily used in clinical applications. For example, to date there are relatively few options available for the diagnosis of breast cancer using easily detectable biomarkers. Overexpression of EGFR, particularly coupled with down-regulation of the estrogen receptor, is a marker of poor prognosis in breast cancer patients. Other known markers of breast cancer include high levels of M2 pyruvate kinase (M2 PK) in blood (U.S. Pat. No. 6,358,683), high ZNF217 protein levels in blood (WO 98/02539), and differential expression of a newly identified protein in breast cancer, PDEBC, which is useful for diagnosis (U.S. patent application No. 20030124543). These biomarkers offer an alternative method of diagnosis, however, they are not widely used. Furthermore, despite the use of a number of histochemical, genetic, and immunological markers, clinicians still have a difficult time predicting which tumors will metastasize to other organs.

The identification of biomarkers is particularly relevant to improving diagnosis, prognosis, and treatment of the disease. As such, there is need in the art to identify alternative biomarkers that can be quickly, easily, and safely detected. Such biomarkers may be used to diagnose, to stage, or to monitor the progression or treatment of a subject with cancer, in particular, an invasive, potentially metastatic stage of the disease.

### SUMMARY OF THE INVENTION

The present invention is based on the discovery that ADAMTS-7 protein is present in urine and ADAMTS-7 expression and activity are up regulated in patients that have breast cancer, prostate cancer, bladder cancer, brain cancer and hepatic cancer. Accordingly, the present invention is directed to methods for prognostic evaluation, methods for facilitating diagnosis of cancers of epithelial origin, and markers for therapeutic efficacy. In particular, the presence of ADAMTS-7 protein detected in biological samples, e.g. urine, predicts the presence of cancer, as ADAMTS-7 protein is not detected at significant levels in healthy individuals. Thus, measuring the presence or absence of ADAMTS-7 in biological samples (e.g. urine or blood) provides a quick, easy, and safe screen that can be used to both diagnose and prognose cancer of epithelial origin, e.g., prostate, breast, hepatic, brain, or bladder cancer, in a patient.

In one embodiment, a method for facilitating the diagnosis of cancer of epithelial origin in a patient is provided. The method comprises obtaining a biological sample from a patient and detecting the presence or absence of ADAMTS-7 (or a fragment thereof) in the biological sample, wherein the presence of ADAMTS-7 is indicative of the presence of cancer of epithelial origin.

In another embodiment, the method comprises measuring the level of ADAMTS-7 present in a test biological sample from a patient and comparing the observed level of ADAMTS-7 with the level of ADAMTS-7 present in a control sample of the same type. Higher levels of ADAMTS-7 in the test sample, as compared to the control sample, is indicative of cancer. Preferably the methods of the invention are used for early detection of cancers of epithelial origin. For example, a patient can be screened by a physician during their annual physicals.

The term “control sample” refers to a biological sample obtained from a “normal” or “healthy” individual(s) that is believed not to have cancer. Controls may be selected using methods that are well known in the art. Once a level has become well established for a control population, array results from test biological samples can be directly compared with the known levels.

The term “test sample” refers to a biological sample obtained from a patient being tested for a cancer of epithelial origin.

Biological samples, for example, can be obtained from blood, tissue (e.g. tumor or breast), serum, stool, urine, sputum, cerebrospinal fluid, nipple aspirates and supernatant from cell lysate. One preferred biological sample is urine.

In one aspect, the cancer of epithelial origin to be diagnosed is breast cancer, basal cell carcinoma, adenocarcinoma, gastrointestinal cancer, such as, for example, lip cancer, mouth cancer, esophageal cancer, small bowel cancer and stomach cancer, colon cancer, liver cancer, bladder cancer, pancreas cancer, ovary cancer, cervical cancer, lung cancer, and skin cancer, such as squamous cell and basal cell cancers, prostate cancer, renal cell carcinoma, and other known cancers that effect epithelial cells throughout the body.

The present invention further contemplates the assessment of ADAMTS-7 levels to monitor the therapeutic efficacy of a treatment regime designed to treat a patient having a cancer of epithelial origin.

In one preferred embodiment, the biological samples are urine samples. However, biological samples of blood, tissue, serum, stool, sputum, cerebrospinal fluid, nipple aspirates, and supernatant from cell lysate can also be used.

In one aspect of the invention, ADAMTS-7 levels present in a test biological sample are measured by contacting the test sample, or preparation thereof; with an antibody-based binding moiety that specifically binds to ADAMTS-7 protein, or to a portion thereof. The antibody-based binding moiety forms a complex with ADAMTS-7 that can be detected, thereby allowing the levels of ADAMTS-7 to be measured.

Antibody-based immunoassays are the preferred means for measuring levels of ADAMTS-7 protein. However, any means known to those skilled in art can be used to assess ADAMTS-7 levels. For example, in some embodiments ADAMTS-7 expression levels are assayed by measuring levels of ADAMTS-7 mRNA transcripts. Alternatively, ADAMTS-7 levels can be assessed by mass spectrometry, including SELDI mass spectrometry. ADAMTS-7 levels can also be assessed by a biological activity assay including, but not limited to, substrate gel electrophoresis (zymography).

In a further embodiment, the invention provides for kits that comprise means for measuring ADAMTS-7 in a biological sample.

In another embodiment, a method to direct treatment of a subject is provided. The method comprises having a subject tested for the presence of ADAMTS-7 in a biological sample obtained from a subject, wherein a clinician reviews the results and if the biological sample is positive for the presence of ADAMTS-7 the clinician directs the subject to be treated accordingly. The test may be performed in the same country where the subject resides or in another country and the results are made available, for example via a Web site, or are transmitted to the clinician.

Other aspects of the invention are disclosed infra.

### BRIEF DESCRIPTION OF THE DRAWINGS

The accompanying drawings, which are incorporated in and constitute a part of this specification, illustrate embodiments of the invention and, together with the description, serve to explain the objects, advantages, and principles of the invention.

FIG. 1 shows the presence of an approximately 190 kDa high molecular weight gelatinase species in urine from a bladder cancer patient by zymography.

FIGS. 2A and 2B shows the partial purification of the approximately 190 kDa high molecular weight gelatinase species from urine of bladder cancer patients. FIG. 2A zymogram. FIG. 2B silver stain gel.

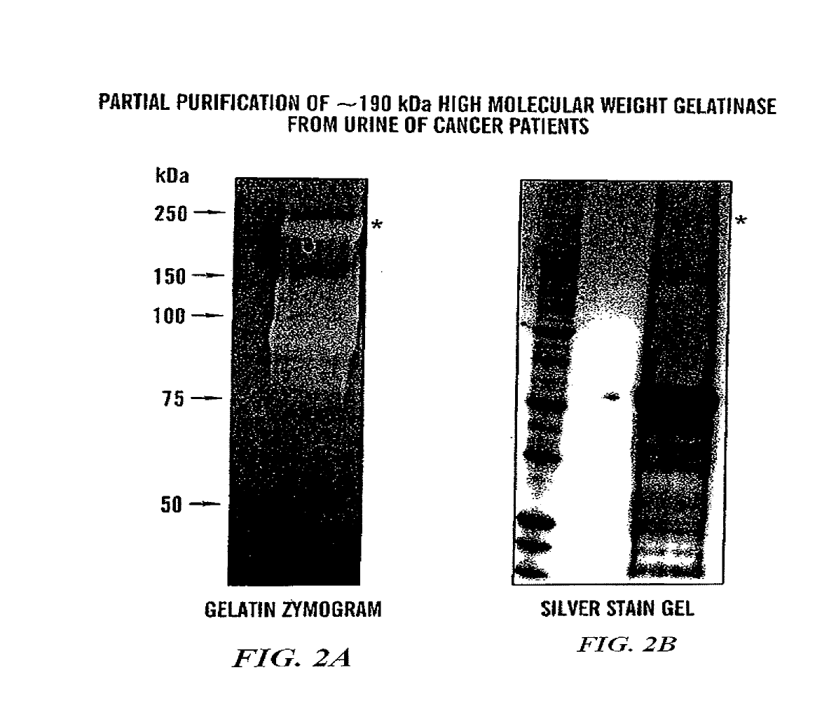

FIG. 3 shows an SDS-PAGE stained with Sypro Ruby Stain of samples enriched for HMW gelatinase species.

FIG. 4 shows the amino acid sequence for ADAMTS-7 (SEQ ID NO:1).

FIGS. 5A and 5B show the detection of ADAMTS-7 by zymography in urine samples from cancer patients and its absence from urine samples obtained from healthy individuals. FIG. 5A, a gelatin zymogram of high MW gelatinase species in urine samples from cancer patients; the first lane represents Molecular weight markers (MW), Lanes indicated 1-9, represent urine samples from individual patients, 50 uls of un-concentrated urine were used, with lane 1 is urine sample from a patient with prostate cancer, lane 2 is a urine sample from a patient with brain cancer, lane 3 is a urine sample from a patient with bladder cancer, lane 4 is a urine sample from a patient with breast cancer, lane 5 is a urine sample from a patient with breast cancer, lane 6 is a urine sample from a patient with hepatic cancer, lane 7 is a urine sample from a patient with hepatic cancer, lane 8 is a urine sample from a patient with breast cancer, and lane 9 is a urine sample from a patient with breast cancer. The arrow points to ADAMTS-7 running at approximately 190 kDa. FIG. 5B shows a parallel zymographic analysis of urine samples from normal age/sex matched controls, patients without cancer. ADAMTS-7 was undetectable in all cases.

FIGS. 6A and 6B show a representative immunoblot staining for ADAMTS-7 protein in urine samples from patients with and without cancer. FIG. 6B, analysis of urine from cancer patients run on a 4-12% gradient SDS-PAGE gel: lane 1 concentrated urine sample from a patient with prostate cancer, lane 2 concentrated urine sample from a patient with prostate cancer, lane 3 concentrated urine sample from a patient with breast cancer, lane 4 concentrated urine sample from a patient with breast cancer, lane 5 concentrated urine sample from a patient with bladder cancer, lane 6 concentrated urine sample from a patient with breast cancer, lane 7 concentrated urine sample from a patient with breast cancer, lane 8 concentrated urine sample from a patient with breast cancer. FIG. 6A shows a parallel immunoblot analysis of concentrated urine samples from normal age/sex matched controls, patients without cancer. The 190 kDa species was detected in urine samples from patients with breast, bladder and prostate carcinomas.

### DETAILED DESCRIPTION OF THE INVENTION

We have discovered that the levels of ADAMTS-7 present in biological samples of patients correlate with the presence, or absence of, cancers of epithelial origin.

As used herein, “cancers of epithelial origin” refers to cancers that arise from epithelial cells which include, but are not limited to, breast cancer, basal cell carcinoma, adenocarcinoma, gastrointestinal cancer, lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body.

The term “aggressive” or “invasive” with respect to cancer refers to the proclivity of a tumor for expanding beyond its boundaries into adjacent tissue (Darnell, J. (1990), Molecular Cell Biology, Third Ed., W. H. Freeman, N.Y.). Invasive cancer can be contrasted with organ-confined cancer wherein the tumor is confined to a particular organ. The invasive property of a tumor is often accompanied by the elaboration of proteolytic enzymes, such as collagenases, that degrade matrix material and basement membrane material to enable the tumor to expand beyond the confines of the capsule, and beyond confines of the particular tissue in which that tumor is located.

The term “metastasis”, as used herein, refers to the condition of spread of cancer from the organ of origin to additional distal sites in the patient. The process of tumor metastasis is a multistage event involving local invasion and destruction of intercellular matrix, intravasation into blood vessels, lymphatics or other channels of transport, survival in the circulation, extravasation out of the vessels in the secondary site and growth in the new location (Fidler, et al., Adv. Cancer Res. 28, 149-250 (1978), Liotta, et al., Cancer Treatment Res. 40, 223-238 (1988), Nicolson, Biochim. Biophy. Acta 948, 175-224 (1988) and Zener, N. Eng. J. Med. 322, 605-612 (1990)). Increased malignant cell motility has been associated with enhanced metastatic potential in animal as well as human tumors (Hosaka, et al., Gann 69, 273-276 (1978) and Haemmerlin, et al., Int. J. Cancer 27, 603-610 (1981)).

As used herein, a “biological sample” refers to a sample of biological material obtained from a patient, preferably a human patient, including a tissue, a tissue sample, a cell sample (e.g., a tissue biopsy, such as, an aspiration biopsy, a brush biopsy, a surface biopsy, a needle biopsy, a punch biopsy, an excision biopsy, an open biopsy, an incision biopsy or an endoscopic biopsy), and a tumor sample. Biological samples can also be biological fluid samples. In one preferred embodiment the biological sample is urine. However, blood, serum, saliva, cerebrospinal fluid, nipple aspirates, and supernatant from cell lysate can also be used.

The present invention also encompasses the use of isolates of a biological sample in the methods of the invention. As used herein, an “isolate” of a biological sample (e.g., an isolate of a tissue or tumor sample) refers to a material or composition (e.g., a biological material or composition) which has been separated, derived, extracted, purified or isolated from the sample and preferably is substantially free of undesirable compositions and/or impurities or contaminants associated with the biological sample.

In a preferred embodiment, the biological sample is treated as to prevent degradation of ADAMTS-7 protein, or ADAMTS-7 mRNA. Methods for inhibiting or preventing degradation include, but are not limited to, treatment of the biological sample with protease or RNAase inhibitors, freezing the biological sample, or placing the biological sample on ice. Preferably, prior to analysis, the biological samples or isolates are constantly kept under conditions as to prevent degradation of ADAMTS-7 protein, or ADAMTS-7 RNA.

As used herein, a “tissue sample” refers to a portion, piece, part, segment, or fraction of a tissue which is obtained or removed from an intact tissue of a subject, preferably a human subject. One preferred tissue sample is mammary tissue.

As used herein, a “tumor sample” refers to a portion, piece, part, segment, or fraction of a tumor, for example, a tumor which is obtained or removed from a subject (e.g., removed or extracted from a tissue of a subject), preferably a human subject.

As used herein, a “primary tumor” is a tumor appearing at a first site within the subject and can be distinguished from a “metastatic tumor” which appears in the body of the subject at a remote site from the primary tumor.

As used herein, “LCIS” refers to lobular carcinoma in situ. LCIS is also called lobular neoplasia and is sometimes classified as a type of noninvasive breast cancer. It does not penetrate through the wall of the lobules. Although it does not itself usually become an invasive cancer, women with this condition have a higher risk of developing an invasive breast cancer in the same or opposite breast.

As used herein, “DCIS” refers to ductal carcinoma in situ. Ductal carcinoma in situ is the most common type of noninvasive breast cancer. In DCIS, the malignant cells have not metastasized through the walls of the ducts into the fatty tissue of the breast. Comedocarcinoma is a type of DCIS that is more likely than other types of DCIS to come back in the same area after lumpectomy, and is more closely linked to eventual development of invasive ductal carcinoma than other forms of DCIS.

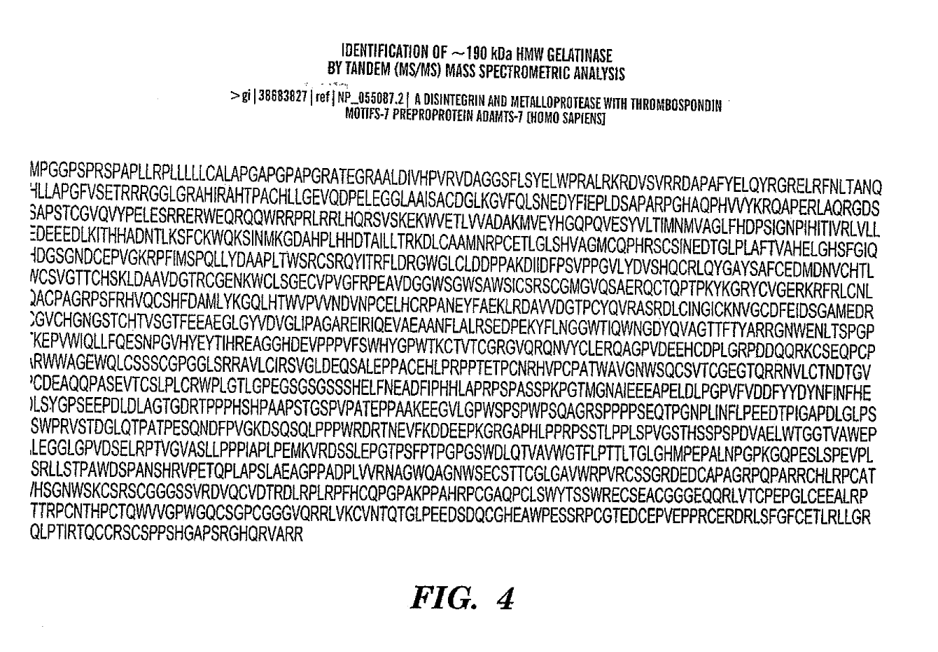

As used herein, “ADAMTS-7” refers to the ADAMTS-7 protein of Genebank accession, protein, NP_{—}055087.2 (Homosapiens) (SEQ ID NO:1) (FIG. 4). ADAMTS-7 is a disintegrin-like and metalloprotease (reprolysin type) with thrombospondin type 1 motif, 7. The term “ADAMTS-7” also encompasses species variants, homologues, allelic forms, mutant forms, and equivalents thereof.

The present invention is directed to methods for facilitating diagnosis of cancer of epithelial origin in a patient. In one embodiment, the method comprises obtaining a biological sample from a patient and detecting the presence or absence of ADAMTS-7 (or a fragment thereof) in the biological sample, wherein the presence of ADAMTS-7 is indicative of the presence of cancer of epithelial origin.

In another embodiment, the methods involve measuring levels of ADAMTS-7 in a test sample obtained from a patient, suspected of having cancer, and comparing the observed levels to levels of ADAMTS-7 found in a control sample, for example a sample obtained from an individual patient or population of individuals that are believed not have cancer. Levels of ADAMTS-7 higher than levels that are observed in the normal control indicate the presence of cancer. The levels of ADAMTS-7 can be represented by arbitrary units, for example as units obtained from a densitometer, luminometer, an activity assay, or an Elisa plate reader.

As used herein, “a higher level of ADAMTS-7 in the test sample as compared to the level in the control sample” refers to an amount of ADAMTS-7 that is greater than an amount of ADAMTS-7 present in a control sample. The term “higher level” refers to a level that is statistically significant or significantly above levels found in the control sample. Preferably, the “higher level” is at least 2 fold greater.

The term “statistically significant” or “significantly” refers to statistical significance and generally means a two standard deviation (2SD) above normal, or higher, concentration of the marker.

For purposes of comparison, the test sample and control sample are of the same type, that is, obtained from the same biological source. The control sample can also be a standard sample that contains the same concentration of ADAMTS-7 that is normally found in a biological sample of the same type and that is obtained from a healthy individual. For example, there can be a standard normal control sample for the amounts of ADAMTS-7 normally found in biological samples such as urine, blood, cerebral spinal fluid, or tissue.

In one aspect of the invention, a secondary diagnostic step can be performed. For example, if a level of ADAMTS-7 is found to indicate the presence of cancer, then an additional method of detecting the cancer can be performed to confirm the presence of the cancer. Any of a variety of additional diagnostic steps can be used, such as mammography (breast cancer), ultrasound, PET scanning, MRI, or any other imaging techniques, biopsy, clinical examination, ductogram, or any other method.

The methods of the invention also are useful for determining a proper course of treatment for a patient having cancer. A course of treatment refers to the therapeutic measures taken for a patient after diagnosis or after treatment for cancer. For example, a determination of the likelihood for cancer recurrence, spread, or patient survival, can assist in determining whether a more conservative or more radical approach to therapy should be taken, or whether treatment modalities should be combined. For example, when cancer recurrence is likely, it can be advantageous to precede or follow surgical treatment with chemotherapy, radiation, immunotherapy, biological modifier therapy, gene therapy, vaccines, and the like, or adjust the span of time during which the patient is treated.

### Measuring Levels of ADAMTS-7

The levels of ADAMTS-7 can be measured by any means known to those skilled in the art. In the present invention, it is generally preferred to use antibodies, or antibody equivalents, to detect levels of biomarker protein. However, other methods for detection of biomarker expression can also be used. For example, ADAMTS-7 expression levels may be monitored by analysis of mRNA transcripts. Measuring ADAMTS-7 mRNA may be preferred, for example when the biological sample is a tumor, or tissue sample.

Methods for assessing levels of mRNA are well known to those skilled in the art. For example, detection of RNA transcripts may be achieved by Northern blotting, wherein a preparation of RNA is run on a denaturing agarose gel, and transferred to a suitable support, such as activated cellulose, nitrocellulose or glass or nylon membranes. Labeled (e.g., radiolabeled) cDNA or RNA is then hybridized to the preparation, washed and analyzed by methods such as autoradiography.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap lipase chain reaction (RT-AGLCR) as described by R. L. Marshall, et al., PCR Methods and Applications 4: 80-84 (1994). One suitable method for detecting ADAMTS-7 mRNA transcripts is described in reference Pabic et. al. Hepatology, 37(5): 1056-1066, 2003, which is herein incorporated by reference in it's entirety.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 454-4610; strand displacement amplification (as described in G. T. Walker et al., Clin. Chem. 42: 9-13 (1996) and European Patent Application No. 684315; and target mediated amplification, as described by PCT Publication WO 9322461.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with haematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Oligonucleotides corresponding to ADAMTS-7 are immobilized on a chip which is then hybridized with labeled nucleic acids of a test sample obtained from a patient. Positive hybridization signal is obtained with the sample containing ADAMTS-7 transcripts. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5; 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescent-labeled cDNA probes are generated. The microarrays capable of hybridizing to ADAMTS-7 cDNA are then probed with the labeled cDNA probes, the slides scanned and fluorescence intensity measured. This intensity correlates with the hybridization intensity and expression levels.

ADAMTS-7 protein levels, or ADAMTS-7 activity, can also be measured, in particular, when the biological sample is a fluid sample such as blood or urine. In one embodiment, levels of ADAMTS-7 protein are measured by contacting the biological sample with an antibody-based binding moiety that specifically binds to ADAMTS-7, or to a fragment of ADAMTS-7. Formation of the antibody-ADAMTS-7 complex is then detected as a measure of ADAMTS-7 levels.

The term “antibody-based binding moiety” or “antibody” includes immunoglobulin molecules and immunologically active determinants of immunoglobulin molecules, e.g., molecules that contain an antigen binding site which specifically binds (immunoreacts with) to ADAMTS-7. The term “antibody-based binding moiety” is intended to include whole antibodies, e.g., of any isotype (IgG, IgA, IgM, IgE, etc), and includes fragments thereof which are also specifically reactive with ADAMTS-7 protein. Antibodies can be fragmented using conventional techniques. Thus, the term includes segments of proteolytically-cleaved or recombinantly-prepared portions of an antibody molecule that are capable of selectively reacting with a certain protein. Non limiting examples of such proteolytic and/or recombinant fragments include Fab, F(ab′)₂, Fab′, Fv, dAbs and single chain antibodies (scFv) containing a VL and VH domain joined by a peptide linker. The scFv's may be covalently or non-covalently linked to form antibodies having two or more binding sites. Thus, “antibody-base binding moiety” includes polyclonal, monoclonal, or other purified preparations of antibodies and recombinant antibodies. The term “antibody-base binding moiety” is further intended to include humanized antibodies, bispecific antibodies, and chimeric molecules having at least one antigen binding determinant derived from an antibody molecule. In a preferred embodiment, the antibody-based binding moiety detectably labeled.

“Labeled antibody”, as used herein, includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS.

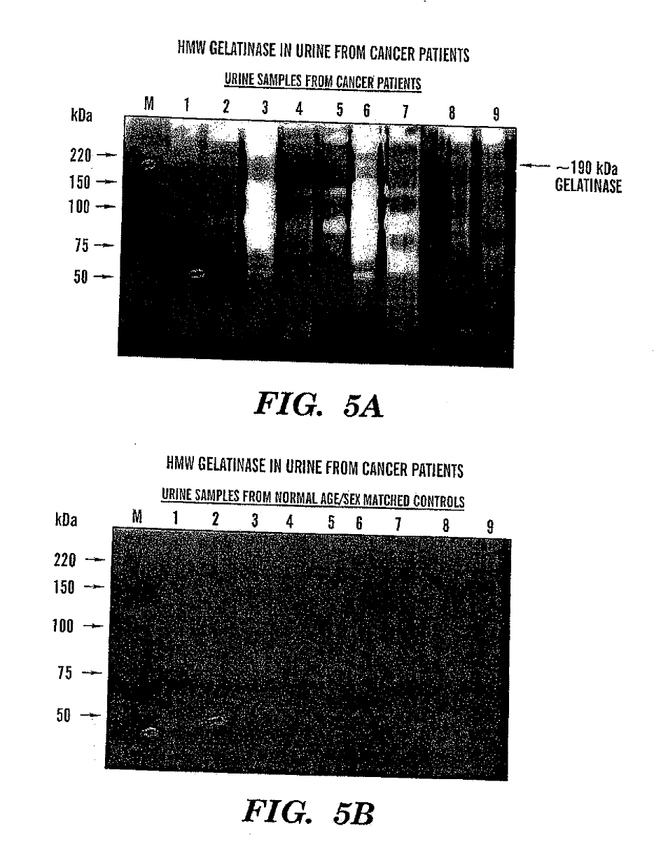

In the diagnostic and prognostic methods of the invention that use antibody based binding moieties for the detection of biomarker levels (e.g. ADAMTS-7 or biomarkers of FIG. 5), the level of biomarker present in the biological samples correlate to the intensity of the signal emitted from the detectably labeled antibody.

In one preferred embodiment, the antibody-based binding moiety is detectably labeled by linking the antibody to an enzyme. The enzyme, in turn, when exposed to it's substrate, will react with the substrate in such a manner as to produce a chemical moiety which can be detected, for example, by spectrophotometric, fluorometric or by visual means. Enzymes which can be used to detectably label the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. Chemiluminescence is another method that can be used to detect an antibody-based binding moiety.

Detection may also be accomplished using any of a variety of other immunoassays. For example, by radioactively labeling an antibody, it is possible to detect the antibody through the use of radioimmune assays. The radioactive isotope can be detected by such means as the use of a gamma counter or a scintillation counter or by audioradiography. Isotopes which are particularly useful for the purpose of the present invention are ³H, ¹³¹I, ³⁵S, ¹⁴C, and preferably ¹²⁵I.

It is also possible to label an antibody with a fluorescent compound. When the fluorescently labeled antibody is exposed to light of the proper wave length, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine.

An antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

As mentioned above, levels of ADAMTS-7 can be detected by immunoassays, such as enzyme linked immunoabsorbant assay (ELISA), radioimmunoassay (RIA), Immunoradiometric assay (IRMA), Western blotting, or immunohistochemistry, each of which are described in more detail below. Immunoassays such as ELISA or RIA, which can be extremely rapid, are more generally preferred. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

### Immunoassays

“Radioimmunoassay” is a technique for detecting and measuring the concentration of an antigen using a labeled (e.g. radioactively labeled) form of the antigen. Examples of radioactive labels for antigens include ³H, ¹⁴C, and ¹²⁵I. The concentration of antigen ADAMTS-7 in a biological sample is measured by having the antigen in the biological sample compete with the labeled (e.g. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

A “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “sandwich ELISA”, an antibody (e.g. anti-ADAMTS-7) is linked to a solid phase (i.e. a microtiter plate) and exposed to a biological sample containing antigen (e.g. ADAMTS-7). The solid phase is then washed to remove unbound antigen. A labeled antibody (e.g. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing antigen (e.g. ADAMTS-7). The antigen-antibody mixture is then contacted with a solid phase (e.g. a microtiter plate) that is coated with antigen (i.e., ADAMTS-7). The more antigen present in the sample, the less free antibody that will be available to bind to the solid phase. A labeled (e.g., enzyme linked) secondary antibody is then added to the solid phase to determine the amount of primary antibody bound to the solid phase.

In a “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or .beta.-galactosidase), or chemical methods (e.g., DAB/Substrate chromagen).

Other techniques may be used to detect the biomarkers of the invention, according to a practitioner's preference, and based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Detectably labeled antibodies that specifically bind to ADAMTS-7 can then be used to assess biomarker levels, where the intensity of the signal from the detectable label corresponds to the amount of biomarker present. Levels can be quantitated, for example by densitometry.

### Mass Spectometry

In addition, biomarkers of the invention may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to these known in the art. For example, in some embodiments a control sample may contain heavy atoms (e.g. ¹³C) thereby permitting the test sample to mixed with the known control sample in the same mass spectrometry run.

In one preferred embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to provide better quantify the amount of the biomolecule present in the first sample. In certain embodiments, the identity of the biomolecules in the first and second sample can also be determined.

In one preferred embodiment, biomarker levels are measured by MALDI-TOF mass spectrometry.

### Other Assays

ADAMTS-7 levels can also be measured by using other biological assays, for example that measure activity, including but not limited to, zymography. Zymography is an assay well known to those skilled in the art and described in Heusen et al., Anal. Biochem., (1980) 102:196-202; Wilson et al., Journal of Urology, (1993) 149:653-658; Hernon et al., J. Biol. Chem. (1986) 261: 2814-2828, Braunhut et al., J. Biol. Chem. (1994) 269: 13472-13479; and Moses et al., Cancer Research 58, 1395-1399, Apr. 1, 1998, which are herein incorporated by reference in their entirety.

### Antibodies

The antibodies for use in the present invention can be obtained from a commercial source. Alternatively, antibodies can be raised against ADAMTS-7, or a portion of the biomarker polypeptide. Methods useful for the production of ADAMTS-7 antibodies are disclosed in U.S. Application. Nos. 2002/0182702; 2003/0212256; 20020110894 and WO 01/11074, which are herein incorporated by reference.

Antibodies for use in the present invention can be produced using standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display library by methods well known in the art. For example, U.S. Pat. No. 5,702,892 (U.S.A. Health & Human Services) and WO 01/18058 (Novopharm Biotech Inc.) disclose bacteriophage display libraries and selection methods for producing antibody binding domain fragments.

### ADAMTS-7 Detection Kit

The present invention is also directed to commercial kits for the detection and prognostic evaluation of a cancer of epithelial origin. The kit can be in any configuration well known to those of ordinary skill in the art and is useful for performing one or more of the methods described herein for the detection of ADAMTS-7. The kits are convenient in that they supply many if not all of the essential reagents for conducting an assay for the detection of ADAMTS-7 in a biological sample. In addition, the assay is preferably performed simultaneously with a standard or multiple standards that are included in the kit, such as a predetermined amount of ADAMTS-7 protein or nucleic acid, so that the results of the test can be quantitated or validated.

The kits include a means for detecting ADAMTS-7 levels such as antibodies, or antibody fragments, which selectively bind to ADAMTS-7 protein, or a set of DNA oligonucleotide primers that allows synthesis of cDNA encoding the protein, or for example, a DNA probe that detects expression of ADAMTS-7 mRNA. The diagnostic assay kit is preferentially formulated in a standard two-antibody binding format in which one ADAMTS-7-specific antibody captures ADAMTS-7 in a patient sample and another ADAM-specific antibody is used to detect captured ADAMTS-7. For example, the capture antibody is immobilized on a solid phase, e.g., an assay plate, an assay well, a nitrocellulose membrane, a bead, a dipstick, or a component of an elution column. The second antibody, i.e., the detection antibody, is typically tagged with a detectable label such as a calorimetric agent or radioisotope.

In one preferred embodiment, the kit comprises a means for detecting levels of ADAMTS-7 in a sample of urine. In a specific embodiment, the kit comprises a “dipstick” with anti-ADAMTS-7 antibodies or fragments, immobilized thereon, which specifically bind ADAMTS-7 protein. Specifically bound ADAMTS-7 protein can then be detected using, for example, a second antibody that is detectably labeled with a calorimetric agent or radioisotope.

In other embodiments, the assay kits may employ (but are not limited to) the following techniques: competitive and non-competitive assays, radioimmunoassay (RIA), bioluminescence and chemiluminescence assays, fluorometric assays, sandwich assays, immunoradiometric assays, dot blots, enzyme linked assays including ELISA, microtiter plates, and immunocytochemistry. For each kit the range, sensitivity, precision, reliability, specificity and reproducibility of the assay are established by means well known to those skilled in the art.

The above described assay kits would further provide instructions for use.

All references cited above or below are herein incorporated by reference.

The present invention is further illustrated by the following Examples.

These Examples are provided to aid in the understanding of the invention and are not construed as a limitation thereof.

### Example I

### Identification of ADAMTS-7 as a High Molecular Weight Gelatinase that is Present in Urine from Cancer Patients

### Identification of Urinary ADAMTS-7.

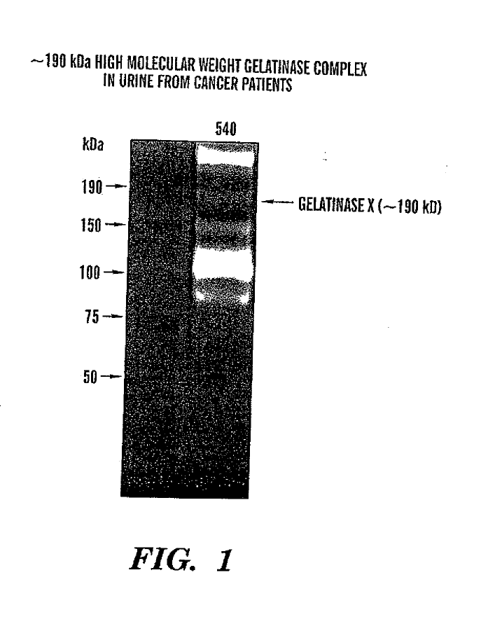

We have identified the approximate 190 kDa high molecular weight gelatinase found in urine samples from a bladder cancer patient (FIG. 1) as ADAMTS-7.

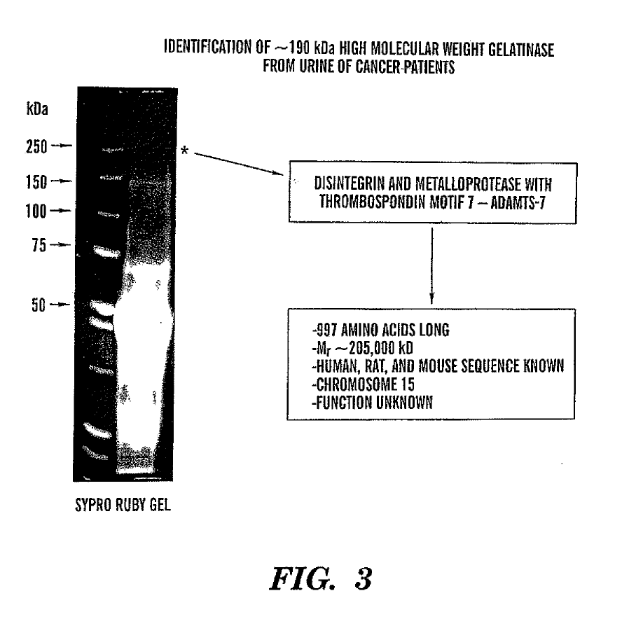

The gelatinase was partially purified using a combination of affinity and ion-exchange chromatography. Samples (from bladder cancer patients) enriched for the high molecular weight gelatinase species were resolved by SDS-PAGE and stained with Sypro Ruby stain (FIG. 3). The protein band of approximately 190 kDa was excised and subjected to in-gel tryptic digest followed by Tandem (MS-MS) mass spectrometric analysis. Mass spectrometric analysis of the approximate 190 kDa gelatinase species indicated the presence of ADAMTS-7 (a disintegrin and metalloprotease (reprolysin type) with thrombospondin type 1 motif, 7; a disintegrin and metalloprotease with thrombospondin motifs, 7 preproprotein) with 1% peptide coverage. Blast analysis and literature search confirmed that the peptides identified by mass spectrometry corresponded to ADAMTS-7 (NP_{—}055087.2).

### Example II

### ADAMTS-7 Expression and Activity are Up Regulated in Patients that have Breast Cancer, Prostate Cancer, Bladder Cancer, Brain and Hepatic Cancer

We tested for ADAMTS-7 activity and expression in patients with and with out cancer. Urine samples were collected from patients with breast cancer, brain cancer, prostate cancer, bladder cancer, and hepatic cancer. 50 uls of un-concentrated urine sample were analyzed by gelatin zymography to detect ADAMTS-7 activity.

For the western blot analysis of ADAMTS-7, the urine samples were concentrated using microcentrifuge spin column (Vivaspin, Vivascience) with a 10 kDa cutoff membrane. All the samples analyzed were normalized for 20 ug total protein. The immunoblot was created from a regular BisTris 4-12% gradient gel, not a zymogram. The ADAMTS-7 antibody used was rabbit polyclonal antibody-RP1-ADAMTS-7 from Triple Point Biologics and is directed to the carboxy-terminus of the protein.

As shown in FIG. 5A, ADAMTS-7 activity was upregulated in patients with that have breast cancer, brain cancer, prostate cancer, brain cancer, bladder cancer, and hepatic cancer. ADAMTS-7 activity was not detected in a parallel zymographic analysis of concentrated urine samples from normal age/sex matched controls, patients without cancer (FIG. 5B).

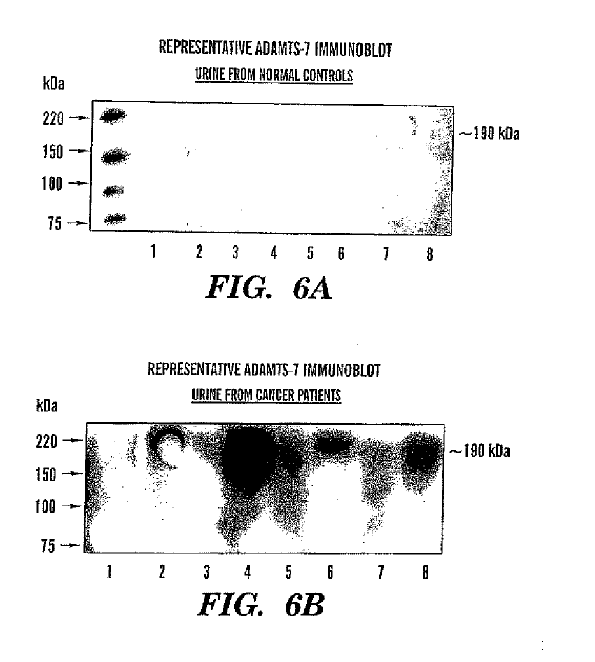

FIGS. 6A and 6B show a representative immunoblot staining for ADAMTS-7 protein in urine samples from patients with and without cancer. As shown in FIG. 6B ADAMTS-7 protein was detected in urine samples from patients with breast, bladder and prostate carcinomas. Parallel immunoblot analysis of concentrated urine samples from normal age/sex matched controls, patients without cancer, did not detect any ADAMTS-7 (FIG. 6A).

The references cited throughout the specification are hereby incorporated by reference.

1. A method for facilitating the diagnosis of a patient for a cancer of epithelial origin comprising:
a. obtaining a biological sample from the patient; and
b. detecting the presence or absence of ADAMTS-7 in the biological sample,
wherein the presence of ADAMTS-7 is indicative of the presence of cancer of epithelial origin.

2. The method of claim 1, wherein said biological sample is selected from the group consisting of blood, tissue, serum, urine, stool, sputum, cerebrospinal fluid, nipple aspirates, and supernatant from cell lysate.

3. The method of claim 1, wherein said biological sample is urine.

4. A method for diagnosing cancer of epithelial origin in a patient comprising:
a. measuring ADAMTS-7 levels present in a test sample obtained from the patient;
b. comparing the level of ADAMTS-7 in the test sample with the level of ADAMTS-7 present in a control sample;
wherein a higher level of ADAMTS-7 in the test sample as compared to the level of ADAMTS-7 in the control sample is indicative of cancer of epithelial origin.

5. The method of claim 4, wherein said test sample and said control sample are selected from the group consisting of blood, tissue, serum, urine, stool, sputum, cerebrospinal fluid, nipple aspirates, and supernatant from cell lysate.

6. The method of claim 4, wherein said test and control samples are urine.

7. The method of claim 4, wherein the cancer of epithelial origin is selected from the group consisting of breast cancer, basal cell carcinoma, adenocarcinoma, gastrointestinal cancer, lip cancer, mouth cancer, esophageal cancer, small bowl cancer, stomach cancer, colon cancer, liver cancer, brain cancer, bladder cancer, pancreas cancer, ovary cancer, cervical cancer, lung cancer, skin cancer, prostate cancer, and renal cell carcinoma.

8. The method of claim 1, wherein the presence or absence of ADAMTS-7 is detected using an antibody-based binding moiety which specifically binds ADAMTS-7 protein.

9. The method of claim 4, wherein the level of ADAMTS-7 is measured by measuring the level of ADAMTS-7 protein.

10. The method of claim 4, wherein the level of ADAMTS-7 is measured by measuring the activity of ADAMTS-7.

11. The method of claim 9, wherein the level of ADAMTS-7 protein is measured by a method comprising the steps of:
a. contacting the test sample, or preparation thereof, with an antibody-based binding moiety which specifically binds ADAMTS-7 to form an antibody-ADAMTS-7 complex; and
b. detecting the presence of the complex, thereby measuring the level of ADAMTS-7 present.

12. The method according to claim 9, wherein the antibody-based binding moiety is labeled with a detectable label.

13. The method according to claim 12, wherein the label is selected from the group consisting of a radioactive label, a hapten label, a fluorescent label, and an enzymatic label.

14. The method according to claim 9, wherein the antibody-based binding moiety is an antibody.

15. The method according to claim 14, wherein the antibody is a monoclonal antibody.

16. A kit for detecting ADAMTS-7 in a urine sample comprising a container for holding a urine sample, and at least one antibody that specifically binds ADAMTS-7.

17. The kit of claim 18, wherein the kit comprises two antibodies that specifically bind to ADAMTS-7, one antibody is immobilized on a solid phase and one antibody is detectably labeled.

18. The kit of claim 18, further comprising directions for use.

19. A method to direct treatment of a subject which comprises having a subject tested for the presence of ADAMTS-7 in a biological sample obtained from the subject, wherein a clinician reviews the results and if the biological sample is positive for the presence of ADAMTS-7 the clinician directs the subject to be treated for cancer of epithelial origin.

20. The method of claim 19, wherein the biological sample is urine.


SEQUENCE LISTING (1 sequence)

SEQ ID NO: 1 - protein, 1686 aa, Homo sapiens
MPGGPSPRSPAPLLRPLLLLLCALAPGAPGPAPGRATEGRAALDIVHPVRVDAGGSFLSYELWPRALRKRDVSVRRDAPAFYELQYRGRELRFNLTANQHLLAPGFVSETRRRGGLGRAHIRAHTPACHLLGEVQDPELEGGLAAISACDGLKGVFQLSNEDYFIEPLDSAPARPGHAQPHVVYKRQAPERLAQRGDSSAPSTCGVQVYPELESRRERWEQRQQWRRPRLRRLHQRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVDGGWSGWSAWSICSRSCGMGVQSAERQCTQPTPKYKGRYCVGERKRFRLCNLQACPAGRPSFRHVQCSHFDAMLYKGQLHTWVPVVNDVNPCELHCRPANEYFAEKLRDAVVDGTPCYQVRASRDLCINGICKNVGCDFEIDSGAMEDRCGVCHGNGSTCHTVSGTFEEAEGLGYVDVGLIPAGAREIRIQEVAEAANFLALRSEDPEKYFLNGGWTIQWNGDYQVAGTTFTYARRGNWENLTSPGPTKEPVWIQLLFQESNPGVHYEYTIHREAGGHDEVPPPVFSWHYGPWTKCTVTCGRGVQRQNVYCLERQAGPVDEEHCDPLGRPDDQQRKCSEQPCPARWWAGEWQLCSSSCGPGGLSRRAVLCIRSVGLDEQSALEPPACEHLPRPPTETPCNRHVPCPATWAVGNWSQCSVTCGEGTQRRNVLCTNDTGVPCDEAQQPASEVTCSLPLCRWPLGTLGPEGSGSGSSSHELFNEADFIPHHLAPRPSPASSPKPGTMGNAIEEEAPELDLPGPVFVDDFYYDYNFINFHEDLSYGPSEEPDLDLAGTGDRTPPPHSHPAAPSTGSPVPATEPPAAKEEGVLGPWSPSPWPSQAGRSPPPPSEQTPGNPLINFLPEEDTPIGAPDLGLPSLSWPRVSTDGLQTPATPESQNDFPVGKDSQSQLPPPWRDRTNEVFKDDEEPKGRGAPHLPPRPSSTLPPLSPVGSTHSSPSPDVAELWTGGTVAWEPALEGGLGPVDSELRPTVGVASLLPPPIAPLPEMKVRDSSLEPGTPSFPTPGPGSWDLQTVAVWGTFLPTTLTGLGHMPEPALNPGPKGQPESLSPEVPLSSRLLSTPAWDSPANSHRVPETQPLAPSLAEAGPPADPLVVRNAGWQAGNWSECSTTCGLGAVWRPVRCSSGRDEDCAPAGRPQPARRCHLRPCATWHSGNWSKCSRSCGGGSSVRDVQCVDTRDLRPLRPFHCQPGPAKPPAHRPCGAQPCLSWYTSSWRECSEACGGGEQQRLVTCPEPGLCEEALRPNTTRPCNTHPCTQWVVGPWGQCSGPCGGGVQRRLVKCVNTQTGLPEEDSDQCGHEAWPESSRPCGTEDCEPVEPPRCERDRLSFGFCETLRLLGRCQLPTIRTQCCRSCSPPSHGAPSRGHQRVARR